(19) United States
(12) Patent Application Publication
(10) Pub. No.: US 2013/0310791 A1
(43) Pub. Date: Nov. 21, 2013

(54) SANITARY OR INCONTINENCE PAD

(71) Applicant: Krzysztof D. Malowaniec, Heidenheim (DE)

(72) Inventor: Krzysztof D. Malowaniec, Heidenheim (DE)

(73) Assignee: Paul Hartmann AG, Heidenheim (DE)

(21) Appl. No.: 13/981,858

(22) PCT Filed: Jan. 4, 2012
(86) PCT No.: PCT/EP2012/050101

§ 371 Date: Jul. 25, 2013

(30) Foreign Application Priority Data
Jan. 25, 2011 (DE) 10 2011 010 269.8

(51) Int. Cl. A61F 13/475; A61F 13/56

(52) U.S. Cl. CPC A61F 13/4752; A61F 13/5616
USPC 604/385.04

(57) ABSTRACT

A sanitary or incontinence pad includes wings which can be folded around the web of an undergarment, wherein the pad has a longitudinal direction and a transverse direction and a front end region in the longitudinal direction and a rear end region in the longitudinal direction, a primary pad part, the longer dimension of which extends in the longitudinal direction and which accommodates an absorption body for body fluids, and further comprising two wings on each longitudinal side of the primary pad part which protrude in the transverse direction starting from the primary pad part such that same form a front and a rear wing pair, wherein the wings of a wing pair can be detachably connected to each other or to an undergarment by means of adhesion zones and are each delimited by a front-side and a rear-side flank, and further comprising barrier elements, which extend and are attached on both sides along longitudinal side edge regions of the primary pad part and which, at the respective distal longitudinal edge, comprise a tensioned elastification means which tries to lift up the barrier element in question, wherein in a front and a rear longitudinal section the respective barrier element, when folded in a flat design, is fixed against an upper face of the primary pad part so as to extend flat such that the barrier element cannot raise up in said longitudinal sections. The properties of the pad during use are improved according to the invention in that, starting from the front or rear end region, the front and rear longitudinal sections of the respective barrier element extend in the longitudinal direction at least to a point at which the neighboring wing has reached half the surface area thereof.

The invention relates to a sanitary or incontinence pad with wings that can be folded about the web of an undergarment, wherein the pad has a longitudinal direction and a transverse direction and an end region which is anterior in the longitudinal direction and an end region which is posterior in the longitudinal direction, with a main pad part which comprises an absorbent body and extends with its longer dimension in the longitudinal direction, and with two wings on each longitudinal side of the main pad part, which wings project from the main pad part in transverse direction so that a front and rear wing pair is formed, wherein the wings of a wing pair are detachably connectable to each other or with an undergarment via adhesive zones and are delimited by a front side flank and a rear side flank, and with barrier elements which extend and are fastened on both sides along longitudinal side border regions of the main pad part, which barrier elements on their distal longitudinal border each have a tensioned elastifying means which seeks to lift up the corresponding barrier element, wherein front and rear longitudinal sections of the respective barrier element are fixed against a top side of the main pad part so as to extend flat in an evenly folded configuration so that the respective barrier element cannot rise up in these longitudinal sections.

Such a sanitary or incontinence pad with two wings on each side is for example already known from WO 96/38110 A or from US 7,056, 311 B2. The primary goal of so-called wings in sanitary or incontinence pads is to detachably fix the pad on the undergarment and thus keep it in a correct position. This is achieved in that the wings detachably interact with each other or with the undergarment via adhesive zones of any desired type, i.e., in particular by means of an adhesive coating or mechanical hook or loop type fastening means. For this, the wings of preferably at least one wing pair, are folded laterally about the longitudinal borders of the web of an undergarment and detachably fixed to one another and/or with the undergarment via the previously mentioned adhesive zones. For this, often one wing pair is sufficient. When the sanitary or incontinence pad is additionally provided with elastified lateral barrier elements, which often are also referred to as cuffs, an additional force acts on the sanitary article as a result of the contracting effect of the elastifying means of the barrier elements, which force not only seeks to lift up the barrier elements at their distal longitudinal border but also to overall exert a deforming force on the lateral barrier elements on the hygiene article. In this regard it is advantageous when the intended arrangement and the detachable fastening of the sanitary or incontinence pad is supported by two wing pairs i.e., by two respective wings which project in transverse direction on each longitudinal side of the main pad part. The wings can—as previously mentioned- be folded about the web of the undergarment or in a manner of speaking be fastened from the inside against a body-facing top side of the undergarment via their previously mentioned adhesive zones. In this context it is noted, that a laterally projecting region of a sanitary or incontinence pad of the type discussed here is referred to as wing when it has an adhesive zone or is configured for folding about the web of an undergarment into a detachably adhering configuration with the corresponding wing on the opposite side of the pad or with the undergarment.

Previously it was assumed, that the presence of wings and in particular the folding of the wings about the web of the undergarment is advantageous especially when interacting with preferably elastified lateral barrier elements (cuffs) because wings in particular during folding exert a transverse pulling force on the barrier elements and thus support or propagate the rising or lifting up of the barrier elements and with this improve the protection against lateral leaking of the hygiene article.

The present invention is based from the object to further refine a sanitary or incontinence pad of the previously described type so that an intended positioning of the pad and its component that are essential for liquid absorption are further optimized during use.

According to the invention, this object is solved in a sanitary or incontinence pad of the mentioned type, in that the front and rear longitudinal sections of the respective barrier element, which front and rear longitudinal sections are fixed so as to extend flat, extend from the front or rear end region in the longitudinal direction at least up to the point at which the neighboring wing has reached half of its surface. The term neighboring wing relates to the wing, which is respectively associated to this respective longitudinal side site and extends outwardly in transverse direction.

In the present invention it was found that the wings can also interfere or even prevent the intended functioning of the cuff or barrier element system by excessively pulling the barrier elements outwardly in an undesired manner in transverse direction which can lead to reduction of the actual elevation z-direction over a liquid absorbing topside of the pad, or the barrier elements are even folded outward in an undesired manner and the distal longitudinal border is pulled in transverse direction outward of a so-called cuff—socket line of the barrier elements. Both has a negative influence on the lateral leaking protection and causes additional forces inside the barrier elements, which are undesired from a point of view of conception of the hygiene article or are not intended and have a negative influence on the use of the hygiene article. In the present invention, it was further recognized that such an influence of the wings can be prevented in that the respective barrier elements in the region of the two-dimensional extent of the rear and front wing pairs are permanently fixed so as to extend flat over a significant portion of the wing pair, i.e., they are in particular folded into one plane and are permanently fixed in this evenly folded configuration against the main pad part so that they cannot rise up but result in a permanently fixed strip. In the region of the longitudinal direction between the wings, the barrier elements or cuffs are provided in such a manner that they can rise up as intended under the influence of their ealstified distal longitudinal border. In this region, the folding of the front and rear wing pairs does then also not have a negative influence as described before, because there, no force component is transferred onto the barrier element system. According to the invention, it was recognized that—as claimed in claim one of the present invention—in case of a minimal extent of the longitudinal section, which is fixed so as to extend flat, of the respective barrier elements, in longitudinal direction from the end regions in the direction towards a transverse center line or towards a center of the sanitary or incontinence pad at least up to a point in longitudinal direction at which the respective wing has reached half (50%) of its surface, satisfactory operating properties of the hygiene article can be achieved. However, it is advantageous when the front and rear longitudinal sections, which are fixed so as to extend flat, of the respective barrier element, extend from the front or rear end region in longitudinal direction at least to a point at which the neighboring wing has reached 60%, in particular 70% of its surface.

In terms of useful distances and/or shapes of the front and rear wing pair, it is advantageous when the front and/or rear longitudinal sections, which are fixed so as to extend flat, extend at most or furthest up to a point at which in a manner of speaking 100% of the surface of the respective wing is reached. The longitudinal sections, which are fixed so as to extend flat, of the respective barrier element would in this case extend in longitudinal direction up to the end of the wing, where also 100% of the wing surface is reached. It is advantageous when the respective fixed longitudinal section extends in the longitudinal direction at most up to a point at which 90%, in particular 80% of the surface of the respective wing is reached.

For delimiting the wings from the main pad part, a straight line or tangent is placed on both sides on the narrowest point of the pad between the front and the rear wing pairs, i.e. at the point at which the contour in transverse direction has the smallest extent. The regions of the pad which in transverse direction are outside of this straight-line or tangent are assigned to the wings or are used for determining the surface or the visual surface of the wings. These applied parallel straight lines or tangents contribute to the determination of the surface or visual surface of the wings in the sense of the present invention, wherein the narrowest point between the front and the rear wing pair separates or distinguishes the front and rear wings from each other. Preferably, a pad according to the invention has exactly 2 wings on each side i.e., a rear and a front wing pair.

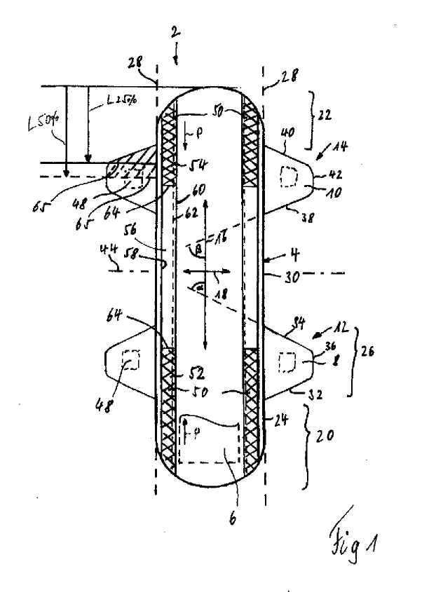

The extent of the longitudinal section, which is fixed so as to extend flat i.e., is not raisable, of the respective barrier element is defined in relation to the front or rear as starting points and then proceeding in the direction toward the transverse center line or the center of the hygiene article. These are the regions of the respective barrier elements, which are shown cross-hatched in the Figures. The increasing surface of the wings is also determined this direction, i.e., also from the front or rear in the direction of the arrows P drawn into FIG. 1. A longitudinal extent L 50% in FIG. 1 indicates a point or a site at which the respective wing has reached about 50% of its two dimensional extent. The surface is delimited by the aforementioned straight line or tangent which is parallel to the longitudinal direction, and is applied to the narrowest point and by a line which runs parallel to the transverse direction of the pad, as can be seen in FIG. 1 (hatched region). It can be seen, the cross-hatched longitudinal section, which is fixed so as to extend flat, of the bordering barrier element extends in longitudinal direction in the direction of the arrow P past the longitudinal extent L 50%, as required according to claim 1 of the present invention.

In the fixed front and rear longitudinal section of the barrier element the barrier element is permanently fixed in particular by adhesive, thermal and/or ultrasound welding sites or combinations to the surface of the main pad part.

According to a further refinement of the invention it is further advantageous when the respective barrier element in the region between the wings is only fixed to the main pad part along a proximal line, the so called Cuff-socket line, so that it can rise up with its distal elastified longitudinal border. In this case, the entire width of the barrier element, i.e., the region between the Cuff-socket line and the distal elastified longitudinal border of the barrier element is available as lateral leak protection, in which the barrier element lifts up from the top side of the main pad part under the influence of its elastified distal longitudinal border. Thereby, well functioning pockets are formed between the barrier elements and the top side of the main pad part.

The fixing of the barrier element along the cuff-socket line occurs preferably by means of welding processes or ultrasound welding and with the welding points produced therewith or by means of adhesive means or combinations of the mentioned methods or means.

As elastifying means for the elastification of the distal longitudinal border of the barrier element, preferably thread or band-shaped elastifying means, such as rubber or polyether polyurethane or polyester polyurethane threads, preferably elastic threads such as Lycra® or Spandex® threads. The elastifying means preferably have a strength of 300-1500 dtex, in particular of 500-1200 dtex, further in particular of 500-900 dtex. As an alternative foamed plastic threads can also be used.

The elastifying means are preferably fixed with a pre-tension of 1.5-6.0, in particular of 2.5-4.5 at the barrier elements.

It is further advantageous when the wings of the front wing pair are arranged within a longitudinal section of the pad with at least 90% of their visible surface, which longitudinal section is at most one third (⅓), in particular at most one quarter (¼) of its overall longitudinal extent. In other words, this means that a predominant portion of the wings of the front wing pair is confined to a longitudinal section of the pad length which makes up at most one third, at most one quarter of the pad length.

It is further advantageous when the front end region in longitudinal direction is at most one third (⅓), in particular at most two sevenths ( 2/7), in particular at least one seventh ( 1/7), in particular at least one sixth (⅙), in particular at least one fifth (⅕) of the overall longitudinal extent of the pad and the wings of the front wing pair are arranged with at least 90% of their visible surface in longitudinal direction outside the front end region.

It is useful and advantageous when the extent of the wings of the front and/or rear wing pair past the narrowest point of the pad between the front and the rear wing pair in transverse direction is 20-70 mm, in particular 20-50 mm, in particular 25-45 mm. In this regard, the extent in transverse direction between the imaginary tangent/straight line that is applied to the narrowest point and the point of the maximal extent of the wing outside of this imaginary line is used for measuring.

It is further advantageous when the visible surface of each wing of the rear wing pair and/or the visible surface of each wing of the front wing pair each occupies a portion of preferably 3-10%, further preferably 3-8%, further preferably 3-7%, further preferably 4-6%, relative to the overall surface of the sanitary or incontinence pad. The surface proportion of the rear wing, which is in this case smaller relative to the overall surface, enables an extent which is in particular sufficient for connecting the wing to the inside of the undergarment while avoiding stiffening resistances as a result of material accumulation in longitudinal direction of the pad.

It is further advantageous when the visible surface of each wing of the front wing pair is respectively 10 to 17 cm², further preferably 11 to 16 cm², further preferably 12 to 15 cm², further preferably 13-14 cm², and/or the visible surface of the rear wing pair is respectively 10-17 cm², further preferably 11-16 cm², further preferably 12-15 cm², further preferably 13-14 cm².

In a preferred embodiment, the overall longitudinal extent of the sanitary or incontinence pad along the longitudinal direction is 150-400 mm, in particular 150-350 mm, in particular 200-350 mm, in particular 200-300 mm, in particular 250-300 mm, in particular 275-290 mm.

In order to realize a most compact configuration and arrangement of the wings in a limited and rather short longitudinal section of the pad, however to still have a surface of the wing which is as great as possible for connection to the undergarment, it is advantageous when the front side flanks and the rear side flanks of the wings of the front and/or rear wing pair extend with the predominant portion of their course at an angle (a, β)>50°, in particular >60°, in particular >65°to the longitudinal direction, wherein the angle is further 60-85°, in particular 60-75°. In the present case, this refers to the smaller of the two cutting angles between the flank direction and the longitudinal direction. In case of such a configuration, the wings become narrower towards the outside, wherein their course is steep, which allows large wing surfaces at a relatively moderate extent of the wing in longitudinal direction.

With regard to the contouring of the wings, the respective front side flanks can for example transition rounded into the rear side flanks. It would also be conceivable however, that a further flank section which is shorter relative to the front side and the rear side flanks is provided between the front side and rear side flanks which further flank section in particular essentially extends in longitudinal direction and into which the flanks preferably transition rounded.

Although it would be conceivable that in the transition between a longitudinal border of the main pad part and a front side or rear side flank of the wings an irregular contour is provided, whereby the formation of a bending or folding line can be given. However, it can also be advantageous when the outer contour of the pad is configured rounded. Further extending on this thought, it is advantageous when a curve radius in the transition between a longitudinal border of the main pad part which essentially extends in the longitudinal direction and the front side flank and/or between the front side flank and the center shorter flank section and/or between the center shorter flank section and the rear side flank and/or between the rear side flank and the longitudinal border of the main pad part which essentially extends in the longitudinal direction of each wing is 5-12 mm, in particular 5-10 mm, in particular 6-9 mm. According to a further inventive idea, which can also be advantageous esthetically, it is proposed to configure the wing contours so that the rear side flanks of the front wing pair and the front side flanks of the rear side wing pair extend straight with the predominant portion of their path.

It can further be advantageous when the front side flanks of the front wing pair and/or the rear side flanks of the rear wing pair extend curved arch-shaped, preferably by forming a outwardly bulged contour, wherein in this case preferably only a very slight arch-shaped curvature with a curve radius of about 100 mm and more, in particular with a curve radius of 120-180 mm, further in particular of 140-160 mm, further in particular of 150-155 mm is provided.

It can further be advantageous when the front side flanks of the front wing pair and the rear side flanks of the rear wing pair extend curved.

The curvature of the front side flank of the front wing pair and/or the rear side flanks of the rear wing pair advantageously contributes to the ergonomic fit of the incontinence or sanitary pad during use. When during use the wings of the front wing pair are folded about the crotch web onto the outside of the under garment and the wings of the rear wing pair are connected against the inside of the undergarment, the curvature of the flanks of the wings supports the flexible adjustment to the three-dimensional shape of the pad. Although the front side flanks of the front wing pair and/or the rear side flanks of the rear wing pair do not have to extend straight but can preferably extend slightly arch-shaped, a tangent can also be placed on the contour, which tangent encloses an angle with the longitudinal direction>50°, in particular >60°, in particular >65°, relative to the longitudinal direction, wherein the angle is further 60-85°, in particular 60-75°.

In a particularly advantageous embodiment of the sanitary or incontinence pad, the front side flanks of the front wing pair and the rear side flanks of the rear wing pair are contoured to correspond to one another, so that they can be brought to align with one another when the pad is folded about an axis which extends in transverse direction (i.e., perpendicular to the longitudinal axis).

It is further advantageous, when the wings of the rear wing pan are arranged within a longitudinal section region of the pad with at least 90% of their visible surface, which longitudinal section region takes up at most one third (⅓), in particular at most one quarter (¼) of its overall longitudinal extent. In other words, this means that a predominant portion of the wings of the rear wing pair is concentrated to a longitudinal section region, which makes up at most one third, at most one quarter of the pad length.

It is further advantageous, when the rear end region in the longitudinal direction makes up at most one third (⅓), in particular at most two seventh ( 2/7), in particular at least one seventh ( 1/7), in particular at least one sixth (116), in particular at least one fifth (⅕) of the overall longitudinal extent of the pad and the wings of the rear wing pair are arranged with at least 90% of their visible surface in longitudinal direction outside of the rear end region.

As an alternative it can be advantageous when the wings of the rear wing pair at the same time form the rear end of the sanitary or incontinence pad.

Particularly advantageously, at least 90% of the visible surface of the rear wing pair are arranged within the rear end region, wherein the rear end region makes up at most one quarter (¼), in particular at most one fifth (⅕) in particular at most one sixth (⅙) of the overall longitudinal extent of the pad.

Here, it is advantageous when the rear side flanks of the rear wing pair extend inwardly toward one another in ergonomic extension of their course and in this way delimit the rear end region of the main pad part. The wings of the rear wing pair thus in a manner of speaking ergonomically transition into the main pad part in one piece so that in longitudinal direction, the main pad part has no further projection extending in rearward direction of the pad.

For fastening on the undergarment, the wings of the sanitary or incontinence pad have adhesive zones. The adhesive zones are provided on the bottom side of the wings. It is further advantageous when the surface of the respective adhesive zone per wing is adjusted to the visible surface of the wing. Preferably, the adhesive zone takes up a surface per wing of 20-50%, further preferably of 20-40% relative to the visible surface of the wing. The adhesive zones are advantageously arranged centered between the front side and the rear side flank of the respective wing.

Also when the pad is fastened by means of the adhesive zones, the short extent in longitudinal direction, in particular in the case of wings of the rear wing pair, is advantageous because large two-dimensional extents of the wings require a greater proportion of adhesive zones to avoid material foldings during use, which in turn would be disadvantageous due to their stiffening effect in these regions.

Further advantageously, the main pad part of the sanitary or incontinence pad is provided with further adhesive zones on the bottom side i.e., on the side which faces the undergarment during use. These further adhesive zones can be arranged as desired in particular in the form of multiple strips that extend in longitudinal direction of the main pad part.

The adhesive zones are preferably provided by an adhesive glue coating, by mechanical hooks and/or loop elements and/or by a combination thereof.

The sanitary or incontinence pads can be distributed to the end user by the manufacturer in evenly spread out condition, in particular stacked. It is also conceivable however, that the pad is folded onto itself about one or more axes that extend in transverse direction, so that its longitudinal extent before use is thereby significantly reduced. Independent of such a folding of the pad about one or multiple transverse axes, the front and/or the rear wings can be folded in onto the top side or the bottom side of the main pad part by the manufacturer, in order to reduce the transverse extent of the product during storage, during distribution for sale and for distribution to the end user. A longitudinal folding axis, which is provided for this purpose, typically extends in longitudinal direction of the pad and can also include border regions of the main pad part outside of the actual wings. However, such a longitudinal fold line, which is made by the manufacturer, preferably extends outside of the absorbent body of the pad.

It can be advantageous when the wings of the front and/or the rear wing pair are folded onto a body-facing top side of the pad by the manufacturer and possibly overlap one another and when an adhesive coating which is then oriented upwards in the respective wings in each wing pair is covered by a single detachable two-dimensional protective element, in particular on paper or film basis, in particular coated with anti-adhesive means, which protective element covers both wings.

The wings of the front and rear wing pairs preferably include or consist of a coversheet material which forms the top side of the sanitary or incontinence pad and of a back sheet material which forms the bottom side of the sanitary or incontinence pad. The wings are preferably free of absorbent materials arranged between the cover sheet and the back sheet material.

The absorbent body preferably extends within the main pad part, and is in particular configured rectangular and extends in longitudinal direction of the pad. It is also conceivable that the shape of the absorbent body in the end regions is adjusted to the contour of the end regions of the main pad part.

For all previously described features, protection is sought for any combination. Further features, details and advantages of the invention result from the drawing and the following description of a preferred embodiment of the sanitary or incontinence pad according to the invention.

In the drawing it is shown in:

FIG. 1 a top view onto a sanitary or incontinence pad in evenly unfolded state;

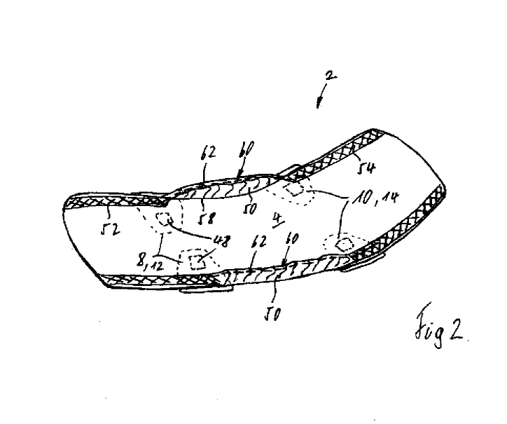

FIG. 2 a perspective schematic view of a sanitary or incontinence pad, which approximately corresponds to FIG. 1 with, outlined wings, which are folded onto the body-averted side of the pad;

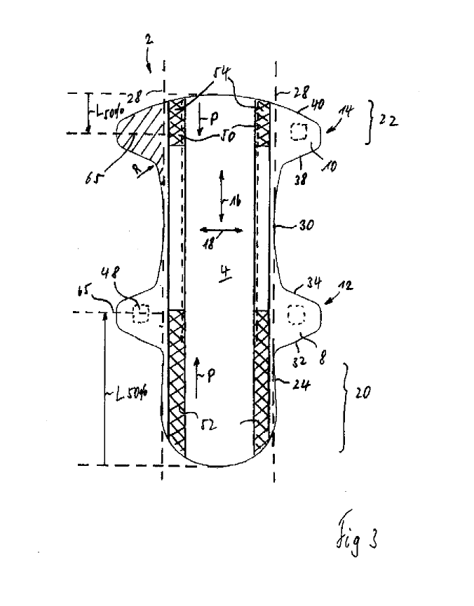

FIG. 3 a top view onto a further embodiment of sanitary or incontinence pad according to the invention in evenly unfolded state;

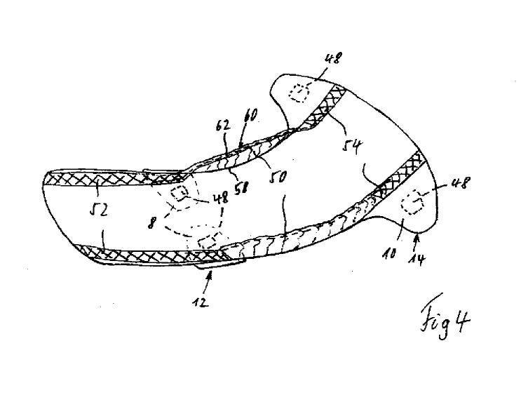

FIG. 4 a perspective schematic view of a sanitary or incontinence pad, which approximately corresponds to FIG. 3, wherein only the wings of the front wing pair are folded onto the body-averted side of the pad;

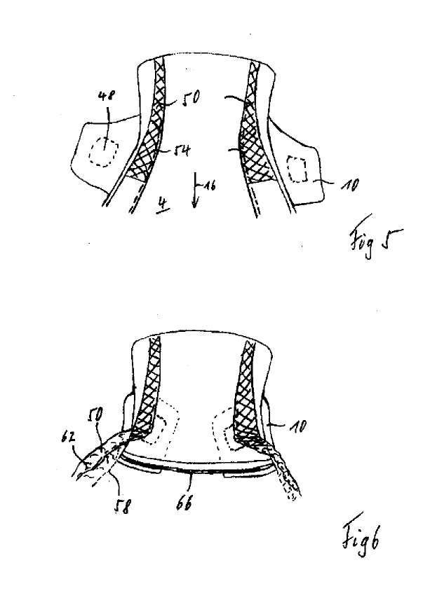

FIGS. 5, 6 schematic perspective representations of a sanitary or incontinence pad according to the invention viewed in longitudinal direction with not folded or folded wings.

FIG. 1 shows a sanitary or incontinence pad, which is overall designated with the reference numeral 2 with a main pad part 4, which in the exemplary shown case includes a rectangular absorbent body 6, and with two wings 8 and 10 on each side. The front wings 8 form a front wing pair 12 and the rear wings 10 form a rear wing pair 14. Reference numeral 16 designates a longitudinal direction and reference numeral 18 a transverse direction of the sanitary or incontinence pad 2, which is perpendicular to the longitudinal direction. Further, a front end region 20 and a rear end region 22 are indicated in FIG. 1. In the front end region 20, no wings are present, but the main pad part 4 delimits the pad with is essentially in longitudinal direction extending longitudinal borders 24. Adjoining this front end region is a longitudinal section 26, which as an example corresponds to at most one quarter of the overall longitudinal extent of the sanitary or incontinence pad, and within which the wings 8 of the front wing pair 12 are arranged with their essentially entire two-dimensional extent. Correspondingly, the wings 10 of the rear wing pair 14 are also provided with the predominant portion of their two dimensional extent in longitudinal direction outside of the rear end region 22, which is not strictly required but only exemplary.

For delimiting the wings 8, 10 from the main pad part 4, a straight line or tangent 28 is placed on each side on the narrowest point 30 of the pad between the front and the rear wing pair 12, 14. The regions of the pad in transverse direction 18 outside of this straight line or tangent 28 are assigned to the wings 8, 10.

The wings 8 of the front side wing pair 12 are delimited by the front side flanks 32 and the rear side flanks 34 and by a shorter flank section 36 which comes to lie between the front side and the rear side flank, which shorter flank section 36 essentially extends in longitudinal direction 16. Also the wings 10 of the rear wing pair 14 are delimited by a front side flank 38 and a rear side flank 40 and by a center shorter flank section 42.

In the exemplary shown case, the flanks 32, 34 or 38, 40 extend straight, which however is not strictly required. They transition rounded into the respective center shorter flank sections 36 or 42.

The flanks 32, 34 of the front wing pair 12 and the flanks 38, 40 of the rear wing pair 14 cross the longitudinal direction 16 at an angle a or b which, also only as an example, is preferably 50°, in particular between 60° and 85°. In this way wings 8, 10 which extend relatively short in the longitudinal direction 16 which provide very good handling during fastening on an undergarment and also offer a high wearing comfort.

The front side flanks 32 of the front wing pair 12 and the rear side flanks 40 of the rear wing pair 14 are contoured to correspond to one another. When the pad is folded onto itself about an axis, which extends in transverse direction, the course of the front side flanks 32 of the front wing pair 12 coincides with the course of the rear side flanks 40 of the rear wing pair 14. Purely exemplary, the flanks 34, 38, which face each other coincide as well.

Further, mechanical or adhesively attaching configured adhesive zones 48 are shown in the wings.

The sanitary or incontinence pad 2 further includes barrier elements 50 which extend on both sides along longitudinal side border regions of the main pad part 4 where they are fastened in a manner to be explained below, and which in the exemplary shown case, extend over the entire longitudinal extent of the pad. They are placed flatly onto the top side of the main pad part 4 in the cross hatched longitudinal section 52 in the front and in the rear 54, and are fixed permanently and so as to extend flat, i.e., over the entire longitudinal section 52 or 54 which is indicated by cross hatching, for example by glue, by embossing or other conventional joining methods.

In the region 56 between the longitudinal sections 52 and 54, the barrier elements 50 which are provided on both sides are only fixed to the main pad part 4 along a proximal line 58, the so called cuff-socket line. The barrier elements 50 which are provided on both sides are elastified at their distal longitudinal border 60 by at least one tensioned elastifying means 62, so that they can rise up from the top side of the main pad part in the region 56 as a result of the tension of the elastifying means 62, in order to form an effective lateral leak barrier.

It can be seen from FIG. 1, that the cross hatched longitudinal sections 52, 54, which have been fixed so as to extend flat, of the barrier elements 50 extend from the end regions 20, 22 in the direction P toward the transverse center line 44 or a center of the pad up to a point or a site 64 at which the neighboring wing 8, 10 has reached at least half of its surface. The point in longitudinal direction or the longitudinal extent at which the respective wing has reached half, i.e. 50% of its surface (in the direction of the arrow P), is designated L 50% in FIG. 1. A point which is associated with 25% of the surface is designated L 25%. The surface is delimited by the aforementioned straight line or tangent 28 applied to the narrowest point 30, which is parallel to the longitudinal direction 16, and by a line 65 which extends parallel to the transverse direction 18 of the pad, as can be seen from FIG. 1 (hatched region).

In this way, it is achieved according to the invention, that the wings 8, 10, in particular when folded onto the body-averted side of the pad 2, influence the intended function of the lateral barrier elements 50 to a minor degree during use.

FIG. 2 shows a perspective view of a sanitary or incontinence pad 2, which can in particular be the sanitary pad shown in FIG. 1, wherein the wings 8, 10 are folded onto the body-averted side of the pad. As a result of the tensioned elastifying means 62 at the respective distal longitudinal border 60 of the barrier elements 50 on both sides, it can be seen how the barrier elements 50 can rise from the top side of the main pad part 4 in the region which in the longitudinal direction is situated between the front and rear wing pair 12, 14 and thus form an effective lateral leak protection. In the respective front and rear longitudinal section 52 or 54 of the barrier elements 50, this is not possible because there the barrier elements are connected with their entire surface with the surface of the main pad part 4.

FIGS. 3 and 4 show a top view and a perspective view of a further embodiment of a sanitary or incontinence pad 2 according to the invention, wherein corresponding reference numerals are used. Essentially, the pad 2 differs according to FIGS. 3, 4 from the pads shown in the FIGS. 1 and 2 in that their flanks 32, 34 or 38, 42 do not extend exactly straight and also the transition of the flanks to lateral longitudinal borders 24 of the main pad part 4 do not exactly extend parallel to the longitudinal direction 16. In addition, the rear wing pair 14 is provided in the rear end region 22 of the pad 2, i.e. as an example such that the respective rear side flank 40 of the rear wings 10 at the same time forms the rear transverse border and with this delimits the rear end region 22 of the pad.

For determining the surface of the wings 8, 10 again a straight line or tangent 28 is placed on the narrowest region in transverse direction 18 between the front and the rear wing pair 12, 14 which straight line or tangent 28 is parallel to the longitudinal direction 16, in order to delimit the surface of the wings 8, 10. This surface is indicated by the hatching of the rear wing 10 on the left hand side in FIG. 3. Again, L 50% designates the point in longitudinal direction 16 starting from the rear end region 22 in the direction of the arrow P at which the respective wing 10 has reached 50% of its surface i.e., delimited by the straight-line or tangent 28 and by the line 65 which is parallel to the transverse direction 18. It can be seen, that the respective longitudinal section 52, 54 of the barrier elements 50 on both sides within which the barrier elements are permanently fastened on the top side of the main pad part 4 so as to extend flat, extend past this value L 50%. Correspondingly L 50% is drawn into the front wing pair.

It can be seen that the flanks 32, 34 or 38, 40 of the front and rear wings 8, 10 transition rounded into the lateral longitudinal borders 24 of the main pad part. When following the course of the flanks, a respective point of a smallest curve radius R.

In the perspective representation according to FIG. 4, the wings 8 of the front wing pair 12 are folded onto the body-averted side of the pad, where they are detachably fastened on the body-averted side of the web of an undergarment. In contrast, the wings 10 of the rear wing pair 14 are not folded around. As a result being arranged in the rear end region 22 of the pad 2 they can also be adhesively detachably fastened with their adhesive zones 48 against the body-facing inside of the undergarment depending on the geometry of the not shown undergarment. Again, it can be seen that the barrier elements can rise up in the region which in the longitudinal direction 16 is situated between the front and rear wing pair 12, 14 essentially without being influenced by the operating condition of the wings 8, 10, i.e., mainly as a result of the tension of the elastifying means 62 that acts in longitudinal direction 16 at the distal longitudinal border 60 of the barrier elements 50. In this region, the barrier elements 50 are preferably connected with the main pad part 4 only along the proximal line 58, the so-called cuff-socket line. However, it is also conceivable that the barrier elements 50 are fixed so as to extend flat, in transverse direction 18 outside of the proximal line 58 over several millimeters. In any case, the fixed region is significantly narrower than the longitudinal section 52 or 54, which is to dimensionally fixed over the entire width.

Finally, FIGS. 5 and 6 show a schematic perspective view of the shape of the barrier elements 50, wherein FIG. 5 shows the state immediately prior to fastening of the sanitary or incontinence pad 2 at an undergarment and FIG. 6 shows the state in which the shown wing pair is folded onto the body-averted side of the pad where it is fastened to a schematically shown undergarment 66. In the representation according to FIG. 5, the barrier elements 50 also lie flatly against the top side of the main pad part 4 in longitudinal direction 16 outside of the longitudinal sections 24 that are fixed so as to extend flat, i.e., in the region between the wings. It can be seen in the representation according to FIG. 6 how the lateral area elements 50 rise up as a result of the tension of their elastifying means 62.

In the case that the geometry or the course of the lateral longitudinal borders 24 is such that the front side flank 32 or the rear side flank 40 of the wings crosses the straight line or tangent 28 and in the further course becomes wider and again in the direction towards its respective longitudinal end, this is not added to the wing surface but this is rather a widened end of the pad without wing function.

What is claimed is:

1.-16. (canceled)

17. A sanitary or incontinence pad having a longitudinal direction, a transverse direction, and front and rear end regions defined along the longitudinal direction, said sanitary or incontinence pad comprising:
a main pad part having a longer dimension and extending with the longer dimension in a longitudinal direction and comprising an absorbent body for absorbing bodily fluids;
front and rear wing pairs, each comprising two wings provided on longitudinal sides of the main pad part and projecting from the main pad part in the transverse direction, said wings of each wing pair provided with adhesive zones and being detachably connectable to one another or with an undergarment via the adhesive zones, each wing being delimited by a front side flank and by a rear side flank; and
barrier elements extending along border regions of respective longitudinal sides of the pad main part and being fastened on the border regions, each one of said barrier elements having a respective distal longitudinal border provided with a tensioned elastifying means, which seeks to lift up the barrier element,
wherein respective front and in a rear longitudinal sections of the barrier elements are fixed against a top side of the main pad part so as to be folded in a flat configuration and to extend flat, thereby preventing rising up of the barrier element in the front and rear longitudinal sections, and
wherein the front and rear longitudinal sections of the barrier elements respectively extend from the front and rear end regions in the longitudinal direction at least to a point at which a neighboring one of the wings has reached half of its surface area.

18. The sanitary or incontinence pad of claim 17, wherein the front and rear longitudinal sections of the respective barrier element extend from the front or rear end region in the longitudinal direction at least up to a point at which the neighboring wing has reached 100%, in particular 90%, in particular 80% of its surface.

19. The sanitary or incontinence pad of claim 17, wherein the respective barrier element in the region between the wings is fixed with the top side of the pad main part only along a proximal line, thereby enabling rising up of the barrier element with its distal elastified longitudinal border.

20. The sanitary or incontinence pad of claim 17, wherein the wings of the front wing pair are arranged with at least 90% of their visible surface within a longitudinal section of the pad which is at most one third, in particular at most one quarter of the overall longitudinal extent of the sanitary or incontinence pad.

21. The sanitary or incontinence pad of claim 17, wherein the front end region in longitudinal direction occupies at most one third in particular at most two seventh in particular at least one seventh, in particular at least one sixth, in particular at least one fifth of the overall longitudinal extent of the pad, and wherein the wings of the front wing pair are arranged with at least 90% of their visible surface in longitudinal direction outside the front end region.

22. The sanitary or incontinence pad of claim 17, wherein an extent of the wings of the front and/or rear wing pair in the transverse direction past a narrowest point of the pad between the front and the rear wing pair is 20-70 mm, in particular 20-50 mm, in particular 25-45 mm.

23. The sanitary or incontinence pad of claim 17, wherein a visible surface of each wing of the rear wing pair and/or a visible surface of each wing of the front wing pair each occupies a proportion of 3-10%, further preferably 3-8%, further preferably 3-7%, further preferably 4-6% relative to an overall surface of the sanitary or incontinence pad.

24. The sanitary or incontinence pad of claim 23, wherein the visible surface of each wing of the front wing is respectively 10-17 cm², further preferably 11-16 cm², further preferably 12-15 cm², further preferably 13-14 cm².

25. The sanitary or incontinence pad of claim 17, wherein the front side and the rear side flanks of the wings of the front and/or rear wing pairs extend with a predominant portion of their course in an angle>50°, in particular >60°, in particular >65°to the longitudinal direction, and wherein the angle is further 60-85°, in particular 60-75°.

26. The sanitary or incontinence pad of claim 17, wherein a flank section is which is shorter relative to the front and rear side flanks is provided between the front side and the rear side flank of a respective one of the wings, said flank section extending in particular essentially in the longitudinal direction, with said front and rear side flanks transitioning rounded into the flank section.

27. The sanitary or incontinence pad of claim 17, wherein a curve radius in a transition between a longitudinal border of the main pad part, which longitudinal border essentially extends in the longitudinal direction, and the front side flank and/or between the front side flank and a center shorter flank section and/or between the center shorter flank section and the rear side flank and/or between the rear side flank and the longitudinal border of the main pad part, which longitudinal border extends essentially in the longitudinal direction of a respective one of the wings is 5-12 mm, in particular 5-10 mm, in particular 6-9 mm.

28. The sanitary or incontinence pad of claim 17, wherein the rear side flanks of the front wing pair and the front side flanks of the rear wing pair extend straight with a predominant portion of their course.

29. The sanitary or incontinence pad of claim 17, wherein the front side flanks of the front wing pair and the rear side flanks of the rear wing pair extend arch-shaped.

30. The sanitary or incontinence pad of claim 17, wherein the front side flanks of the front wing pair and the rear side flanks of the rear wing pair are contoured so as to correspond to one another, and to be aligned with each other when folding the pad about an axis which extends in the transverse direction.

31. The sanitary or incontinence pad of claim 17, wherein the rear side flanks of the rear wing pair extend inwardly toward one another in an ergonomic extension of their course, thereby delimiting the rear end region of the main pad part.

32. The sanitary or incontinence pad of claim 17, wherein the wings of the front and/or the rear wing pair are folded by the manufacturer onto a body-facing top side of the pad and in particular overlap one another, and wherein an adhesive coating which then faces upwards is covered in the respective wings in each wing pair by a single detachable two-dimensional protective element, in particular on paper or foil basis, in particular coated with anti adhesive means, which protective element covers both wings.
